(19) United States
(12) Patent Application Publication
(10) Pub. No.: US 2006/0205914 A1
(43) Pub. Date: Sep. 14, 2006

(54) Method for producing polyesterols having a reduced reactivity

(75) Inventors: Mirko Kreitschmann, Aachen (DE); Udo Rotermund, Ortrand (DE); Peter Fritsch, Grossraeschen (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellshaft, Ludwigshafen (DE)

(21) Appl. No.: 10/548,482

(22) PCT Filed: Feb. 24, 2004
(86) PCT No.: PCT/EP04/01801

§ 371 Date: Sep. 9, 2005

(30) Foreign Application Priority Data
Mar. 12, 2003 (DE) 103 11 121.2

(51) Int. Cl. C08G 18/42; C07C 69/34

(52) U.S. Cl. 528/83; 560/198

(57) ABSTRACT

Polyesterols having reduced reactivity are prepared by catalytic polycondensation of dicarboxylic acids/dicarboxylic acid derivatives and H-functional substances using tin catalysts by a process wherein the polyesterols are brought into contact with oxygen after the end of the esterification reaction. The polyesterols themselves obtained by this process are used for the preparation of PU.

The present invention relates to a process for the preparation of polyesterols by catalytic polycondensation of dicarboxylic acids/dicarboxylic acid derivatives and H-functional substances using tin catalysts having a low reactivity with respect to isocyanates.

The preparation of polyesterols and the use of such products in polyurethane chemistry have long been known and are widely described in general, these products are prepared by esterifying dicarboxylic acids and polyfunctional alcohols. An overview of the preparation of polyesterols and the processing thereof to polyurethanes (also referred to below as PU), in particular PU foams, is given, for example, in Kunststoff-Handbuch, Volume VII, Polyurethane, 1st edition, 1966, edited by Dr. R. Vieweg and Dr. A. Hochtlen, and 2nd edition, 1983, and 3rd edition, 1993, edited by Dr. G. Oertel (Carl HanserVerlag, Munich).

The usual reaction of aromatic and/or aliphatic dicarboxylic acids, such as adipic acid or phthalic acid, with difunctional and/or trifunctional alcohols, such as ethylene glycol and its higher homologs, diethylene glycol, propylene glycol and its higher homologs, dipropylene glycol, butanediol, neopentylglycol, hexanediol, trimethylolpropane or glycerol, in the presence or absence of metal catalysts, leads to polyesterpolyols having a broad molecular weight range and a wide range of applications.

Hydroxy compounds, such as polyesterols, react with isocyanates with formation of PU. The polymer formation and preparation of products having optimum and especially constant properties should take place in an advantageous period. In many applications, a defined reaction rate between polyesterols and isocyanates is required for the formation of very specific product properties or a reproducible processing method. Thus, too high a reaction rate may lead to a rapid increase in the viscosity (possibly to solidification of the product), which may give rise to difficulties during the processing (foam collapse in the case of flexible foams, solidification of the product in TPU production, casting time in the case of cast elastomers). A complication for the processor is the fact that the reactivity of the polyesterols may vary depending on the preparation process and hence the processing method frequently has to be modified as a function of the reactivity. For the processor, too high a reactivity is more difficult to regulate than too low a reactivity. In the case of too low a reactivity, the process can be easily adjusted by adding known PU catalysts.

It is furthermore known that the catalysts used for accelerating the esterification process can influence the PU reaction. For certain PU applications, it is therefore necessary to choose a small amount of catalyst, which however leads to long batch run-times, or to modify the reaction rate by means of additives. According to the prior art, inhibitors can be used for adjusting the reaction rate between H-active compounds and isocyanates. Such inhibitors are described, for example, in the Kunststoff-Handbuch cited above, e.g. hydrochloric acid, benzoyl chloride and p-toluenesulfonic acid. P-containing compounds, as described in the article by B. Fortunato, A. Munari, P. Manaresi and P. Monari in Polymer Vol. 35, Issue 18, page 4006, can complex the esterification catalyst and thus also slow down the subsequent PU reaction. The generally acidic compounds can, however, lead to an undesired deterioration in the stability of the product to hydrolysis. In DE-A-2451727, DD 148882 and DD 148460, aldehydes, ketones or β-diketones are therefore used for reducing the reactivity. However, additives of this type frequently have a certain volatility and can thus contribute to the known fogging phenomenon. In general, it is possible for low molecular weight substances also to migrate to the surface and contribute there to a deterioration in the surface properties of the PU.

A further possibility for reducing the reactivity of polyesterols is to deactivate the esterification catalysts used by hydrolysis. Thus, DD 126276 describes a process for adjusting the reactivity by adding from 0.001 to 1.5% by mass of water. A disadvantage of this process is that removal of the water is necessary for some applications (prevention of the foam reaction). Increased water contents moreover lead to an acceleration of the hydrolysis and hence to a reduced shelf-life during the storage of the polyesterol.

It is an object of the present invention to provide a simple and economical process which makes it possible to prepare polyesterols having a low reactivity with respect to isocyariates. In the preparation, short batch run-times should be achieved. Disadvantages which arise as a result of the addition of additives, such as a reduction of the shelf-life, a reduction of the stability to hydrolysis, migration effects and troublesome color effects, should not occur.

We have found that this object is achieved, surprisingly, if the polyesterols prepared by catalytic polycondensation of dicarboxylic acids/dicarboxylic acid derivatives and H-functional substances using tin catalysts are brought into contact with oxygen after the end of the esterification reaction.

The present invention accordingly relates to a process for the preparation of polyesterols of reduced reactivity by catalytic polycondensation of dicarboxylic acids/dicarboxylic acid derivatives and H-functional substances using tin catalysts, wherein the polyesterols are brought into contact with oxygen after the end of the esterification reaction.

The present invention furthermore relates to the polyesterols themselves which are obtained by this process and to the use of said polyesterols for the preparation of PU.

It was surprising and in no way foreseeable that the reactivity of the polyesterol with respect to isocyanates can be reduced by bringing into contact with oxygen, in particular atmospheric oxygen, without prior hydrolysis of the organometallic catalyst.

Rather, it was to be expected that a reduction in the reactivity of the polyester would be achievable only by hydrolysis of the homogeneously dissolved catalyst.

As a result of bringing the reaction mixture into contact with oxygen and the associated reduction in the reactivity of the polyesterol with respect to isocyanates, it became possible to operate with high catalyst contents in the process for the preparation of the polyesterols and hence to achieve shorter batch run-times. At the same time, the prepared polyesterols have low metal contents, which is desirable when used in PU preparation in many applications. For example some tin compounds are suspected of being harmful to health.

The polyesterols prepared by the novel process have a low reactivity with respect to isocyanates, but not the stated disadvantages, such as a reduction of the shelf-life, a reduction of the stability to hydrolysis, migration effects and troublesome color effects. By reducing the reactivity of the polyesterol, it is made easier for the processor to establish the optimum process parameters. An improved and constant product quality is achieved during the processing.

The novel preparation of the polyesterols is effected by catalytic polycondensation of dicarboxylic acids/dicarboxylic acid derivatives and H-functional substances, preferably at from 150 to 280° C, in particular from 200 to 250° C., with the use of tin catalysts under atmospheric pressure or reduced pressure. If required, the water of reaction can be separated off by passing in propellants, such as nitrogen.

Aromatic and/or aliphatic dicarboxylic acids or dicarboxylic acid derivatives, for example dicarboxylic anhydrides, and preferably difunctional or trifunctional and/or higher-functional alcohols, serve as starting materials for the novel process.

For example, adipic acid, phthalic acid, isophthalic acid and terephthalic acid, the latter generally in the form of their anhydrides, and also oxalic acid, succinic acid, glutaric acid, azelaic acid and/or sebacic acid and others, are used as aliphatic and/or aromatic dicarboxylic acids. Adipic acid, isophthalic acid, phthalic acid and terephthalic acid in the form of pure acids and/or in the form of anhydrides and/or derivatives of these acids are preferably used. The compounds can be used individually, as any desired mixtures with one another or as a mixture with further aromatic and/or aliphatic acids.

The H-functional substances are used preferably in a molar excess of from 1.01:1 to 5:1, particularly preferably from 1.03:1 to 2:1, based on the dicarboxylic acids/dicarboxylic acid derivatives used, in order to obtain polyesterols having terminal hydroxyl groups.

In particular, aliphatic-alcohols, such as ethylene glycol, propylene glycols or glycols having a larger number of chains, diglycols and/or polyols, in particular polyetherpolyols, polyesterpolyols or polyetheresterpolyols, are used as H-functional substances. These can be used individually or in the form of any desired mixtures of a plurality of alcohols with one another or with trifunctional and/or higher-functional alcohols and/or polyols, e.g. trimethylolpropane and glycerol.

Amines, for example alkyl- or arylamines, or aliphatic and/or aromatic compounds which comprise a plurality of amino groups, such as ethylenediamine, diethylenetriamine, triethylenetetramine and tetraethylenepentamine, can also be used as H-functional substances.

Glycols, glycerol, trimethylolpropane and/or pentaerythritol are particularly preferred. The reaction is accelerated by adding tin catalysts customary for the esterification. Sn(II) compounds, for example tin(II) 2-ethylhexanoate, SnCl₂ and SnO, but also Sn(IV) compounds, such as dibutyltin dilaurate (DBTL), are preferably used according to the invention.

The novel process proves particularly advantageous when tin(II) catalysts are used, tin(II) 2-ethylhexanoate or tin(II) chloride being employed in a particularly preferred variant of the process.

According to the prior art, the amount of tin catalysts added is usually from 0.01 to 1 000 ppm, in particular from 0.1 to 50 ppm, based in each case on the prepared polyesterol. According to the novel process, the reactivity of catalyzed polyesterols can be reduced, leading to improved processibility in certain PU preparation processes. According to the novel process, it is therefore possible to use higher catalyst concentrations, which accelerates the reaction and shortens the reaction cycle.

According to the invention, up to 1 000 ppm, particularly preferably from 1 to 1 000 ppm, of catalyst are advantageously used.

The reactivity of the polyesterols with respect to isocyanates is reduced, according to the invention, by bringing the polyesterols into contact with oxygen after the end of the esterification reaction.

The oxygen can be used in the form of pure oxygen or as a mixture with other gases, such as nitrogen or other inert gases, such as helium, neon or argon.

Atmospheric oxygen is preferably used. The contact with air can be ensured, for example, by passing in dry compressed air or passing in a lean air mixture or by storing the polyesterol mixture in the air. The oxygen is then absorbed via the polyesterol/air interface. Lean air is understood as meaning inert gas/air mixtures having an oxygen content which is lower than that of air. The oxygen content is preferably less than 12% by volume. The inert gas is generally nitrogen.

Advantageously, the polyesterol is brought into contact with oxygen or mixtures thereof or with the air
in a polyester reactor during the reaction, preferably after the end of the reduced-pressure phase (variant 1) and/or
in the storage tank of the prepared polyesterol at the manufacturer's or the customer's premises (variant 2) and/or
in the tanker vehicles which are used for transporting the polyesterols, in particular by purging the tanker vehicles with dry compressed air (variant 3).

Variant 1 has the advantage that existing means for the polyester reactor can be used for passing in gases which as a rule are present. The feeding of oxygen can alternatively be effected in a particularly simple manner by breaking the vacuum with atmospheric oxygen.

Variant 2 has the advantage that the absorption of the air at the polyol/air interface can take place in the storage tank. No additional apparatuses are required for passing in gases. Expensive means for providing an inert atmosphere in the tanks can be dispensed with. By thorough mixing of the tank content, for example by stirring or circulation, the absorption of air can be increased and hence the reduction of the reactivity can be regulated.

Variant 3 has the advantage that the installations required for purging the tanker vehicle can be used for passing in oxygen.

Depending on the valency of the tin catalyst used and on the concentration and amount of the atmospheric oxygen or air mixture brought into contact with the polyol, the reactivity of the polyesterol can be reduced to different extents. The exact degree of reduction is dependent in practice on the storage temperature, the duration of exposure of the polyesterol to air and the degree of mixing of the tank or reactor content and must be established individually as a function of the conditions prevailing in practice.

The novel process proves particularly advantageous if the polyesterol is brought into contact with air by passing in the latter in the form of dried compressed air or lean air (variant A) and/or by absorption of oxygen by storage of the polyesterol under atmospheric conditions (variant B).

In a preferred embodiment of variant A, the oxygen is passed in in the form of compressed air, lean air or a mixture of oxygen and an inert gas. The duration of passing in is from 1 to 1 500, advantageously from 5 to 500, particularly preferably from 15 to 350, minutes. When the bringing into contact with oxygen is complete, a time span of at least 1 minute, advantageously at least 15, particularly preferably at least 60, minutes should be allowed between the end of passing in and the further processing to give the PU.

In a preferred version of variant B, the storage of the polyesterol in the air is effected for at least 5, advantageously at least 15, particularly preferably at least 60, minutes. If the polyesterol is handled according to the invention using variant B, the further processing to give the polyurethane can follow immediately.

After having been brought into contact with oxygen, the polyesterols prepared according to the invention can be stored in the liquid state at at least 40° C., preferably from 50 to 95° C.

Traditionally, the further storage is effected at reduced pressure or under N₂. It can also be effected under oxygen (mixtures) or air. However, it is advisable to degas the polyesterol before the step involving the processing of the polyesterol to the polyurethane, since a harmful effect during the processing to the polyurethane at relatively high temperatures is attributed to the oxygen.

If required, the polyesterols prepared according to the invention can moreover be subjected to a conventional aftertreatment.

The novel polyesterols typically have a lower reactivity with respect to isocyanates than comparably catalyzed polyesterols which were not subjected to the novel treatment. Furthermore typical of the novel polyesterols is that, after the novel treatment of the polyesterol with oxygen/air, the tin content of the polyesterol which can be determined by polarography is lower than the tin content which can be determined by ICP-AAS (inductively coupled plasma atomic absorption spectroscopy) or XFA (X-ray fluorescent analysis).

Sn(II) compounds, such as SnC₂ or Sn(II) octanoate, and Sn(IV) compounds, such as DBTL, can be converted into an ionic form by strong acids and quantitatively and qualitatively detected by means of the polarography method. After the novel treatment of the tin-catalyzed polyesterols, an oxidation of the catalyst from Sn(II) to Sn(IV) species probably takes place and subsequent reaction with traces of water (hydrolysis) forms aggregates which represent precursors of SnO₂. Sn(IV) compounds can likewise aggregate under the conditions described according to the invention. These aggregates can no longer be converted into an ionic form with strong acids and are no longer detected by means of polarography. However, the total tin content which is determined by the ICP-AAS or XFA methods remains virtually the same before and after the introduction of air since these methods are not sensitive to the oxidation state of the tin but only to the total amount of tin. According to the invention, the tin content detectable by means of polarography is therefore below the tin content determined by ICP-AAS or XFA.

Sn(IV) catalysts, such as dibutyltin dilaurate or dibutyltin oxide, lose their polarographic activity substantially more slowly than Sn(II) catalysts after they have been brought into contact with oxygen/air according to the invention. Dialkyltin compounds can aggregate substantially only to polymeric chains and not to three-dimensional objects. There is therefore a smaller difference between the tin contents detected by polarography and the tin contents detected by ICP-AAS, AES (atomic emission spectroscopy) or XFA. Such catalysts are therefore not so suitable for the preparation of polyesterols which are to have a low reactivity with respect to isocyanates.

The starting solution used in the polarography and apparatus setup are described below:

Polarographic determination of tin catalysts

Apparatuses:
VA processor 746 with VA standard 694, from Metrohm
Electrode: MME as dropping mercury electrode (DME)
Reference electrode: Ag/AgCl
Salt bridge: LiCl saturated in ethanol

Starting solution:

Determination of the tin content of tin(II)-catalyzed polyesterols
0.2 mol of methanesulfonic acid in a toluene/methanol mixture (1:1% by volume/% by volume),
standard solution of the pure tin(II) catalyst, e.g. tin(II) 2-ethylhexanoate, in ethanol, concentration should correspond roughly to ten times the expected content of the sample.

Determination of the tin content of tin(IV)-catalyzed polyesterols
0.2 mol of methanesulfonic acid in a mixture of dimethylformamide (DMF)/water (90:10% by volume/% by volume),
standard solution of the pure tin(IV) catalyst, e.g. dibutyltin dilaurate, in DMF/water (9:1), concentration should correspond roughly to ten times the expected content of the sample.

Measurement conditions:

Differential pulse polarography (DP polarography), corresponding to the basic settings on the VA 746 for the DP method

Procedure:

10 ml of the starting solution are initially taken in the polarography vessel. 2.5 g of polyesterol sample are weighed accurately to 1 mg into a 25 ml volumetric flask and dissolved in a 1:1 (% by volume/% by volume) mixture of toluene and methanol and made up to the mark.

10 ml of this solution are pipetted into the starting solution in the polarography vessel. After deaeration with nitrogen (5 minutes), the polarogram is recorded in the range from −100 to −800 mV. The peak between −300 and −600 mV, which corresponds to the tin compound, is evaluated under standard conditions. 0.1 ml of the standard solution of tin octanoate is then added to the measuring solution and polarography is carried out again. A second increase in concentration is carried out analogously.

The sample concentration is calculated from the currents of the sample measurement and the standard additions by the usual methods as described, for example, in Gunter Henze, Polarographie und Voltametrie—Grundlagen und analytische Praxis, Springer Verlag Berlin, 2001.

Reactivity Test

The reactivity number (RN) is determined from the average slope of the temperature-time curve of the urethane reaction up to 95° C., in K/s.

A formulation consisting of the following is used for this purpose:

11222/OH number g (0.1 mol) of the polyesterol to be tested,

20 g (0.22 mol) of 1,4-butanediol and

80.1 g (0.32 mol) of 4,4′-diphenylmethane diisocyanate.

The determination is based on the reaction of the polyesterol in the presence of chain extenders with diisocyanate according to the above formulation and monitoring of the reaction temperature of the urethane formation reaction over the time until a reaction temperature of 95° C. is reached. For this purpose, the components heated to 60° C. (A component: polyesterol and 1,4-butanediol, B component: diphenylmethane diisocyanate) are mixed with one another at 60° C. (starting point of the reaction) and the time At until 95° C. has been reached is measured. RN is defined as follows:
RN=ΔT/Δt=(368 K−333 K)/(Δt)=35 K/Δt[K/s],

where Δt is the duration from the starting point of the reaction until 95° C. is reached. The lower the RN, the less reactive is a polyesterol.

The working examples which follow illustrate the invention without restricting it.

### EXAMPLE 1

A polyesterol was prepared from 5731 kg of adipic acid (ADA) and 3 923 kg of 1,4-butanediol (B14) in an esterification reactor at 240° C. while distilling off the resulting condensation water. At an acid number (AN) of about 5 mg KOH/g, 10 ppm of tin(II) 2-ethylhexanoate were added (corresponds to 2.8 ppm of Sn metal). After an AN of <1 mg KOH/g had been reached, this polyesterol was forced under nitrogen into a storage tank (OH number of the polyesterol: 46 mg KOH/g).

A sample (about 5 kg in a tinplate bucket) of this polyesterol batch was taken from the storage tank and the reactivity was determined about 1 hour later. The reactivity number RN was 0.24 K/s. The tin content was determined by means of polarography and ICP-AAS. The Sn content was 2 ppm in the polarography, 3 ppm having been determined by means of ICP-MS (theoretical value 3 ppm).

The residual sample amount was stored at 95° C. in a drying oven for 3 days under atmospheric conditions. The reactivity was then measured again. The reactivity number decreased to 0.19 K/s. The polarographic tin content was <1 ppm and the tin content measured by ICP-AAS was once again 3 ppm.

On the same day, a further sample was taken from the storage tank blanketed with nitrogen. The reactivity number was once again 0.24 K/s and the polarographic tin content was 2 ppm, while the tin content determined by ICP-AAS corresponded to 3 ppm.

The example shows that the storage conditions have a significant effect on the polyesterol reactivity. On blanketing with nitrogen, the ester scarcely loses its initial reactivity, if at all. On storage in the air, the reactivity number decreased by 80% of the original value. The decrease in the reactivity correlates with a decreasing content of polarographically detectable tin. Typical of the ester treated according to the invention is that the tin content determined by ICP-AAS is higher than the polarographically detectable tin content.

### EXAMPLE 2

### Example Comparable to Example 3

75 ppm of tin(II) 2-ethylhexanoate (corresponds to 22 ppm of Sn) were added shortly before the end of the reaction (AN about 2 mg KOH/g) to a polyesterol obtained from 59.36 kg of ADA and 40.6 kg of B14. The reactivity of the sample was determined immediately after the end of the reaction. Dry compressed air (50 l/h) was passed into about 4 kg of the sample in a 5 l three-necked flask having a stirrer and a gas inlet tube at 95° C., and the reactivity number was determined at intervals of 24 hours.

TABLE

| | Storage | Storage | | Sn | |
|---|---|---|---|---|---|
| Esterol | method | time | RN | (polarography) | Sn (AAS) |
| VP9186 | Introduction | 0 h | 0.95 K/s | 20 ppm | 22.0 ppm |
| | of air | 24 h | 0.19 K/s | <1 ppm | 22.2 ppm |
| VP9186 | Introduction | 0 h | 0.90 K/s | 19 ppm | 22.3 ppm |
| | of air | 23 h | 0.17 K/s | <1 ppm | 22.5 ppm |

These experiments show that the reactivity of the polyesterol decreases as a result of being brought into contact with air. Typical of the esterol is that the polarographically detectable tin content is as high as the tin content determined by means of ICP-AAS.

### EXAMPLE 3

75 ppm of tin(II) 2-ethylhexanoate (corresponds to about 22 ppm of Sn) were added shortly before the end of the reaction (AN about 2 mg KOH/g) to a polyesterol obtained from 59.36 kg of ADA and 40.6 kg of B14. The reactivity of the sample was determined immediately after the end of the reaction. 4 kg of the sample was stored in a flask under reduced pressure (10 mbar) at 95° C. The reactivity of the samples was determined at regular intervals.

TABLE

| | Storage | Storage | | Sn | |
|---|---|---|---|---|---|
| Ester | method | time | RN | (polarography) | Sn (AAS) |
| VP9186 | Reduced | 2 h | 0.88 K/s | 19 ppm | 22.1 ppm |
| | pressure | | | | |
| | | 20 h | 0.97 K/s | 20 ppm | 22.3 ppm |

These experiments show that the reactivity of polyesterols treated according to the invention does not change in comparison with example 3. The polarographically determined tin content corresponds to the tin content which was determined by means of ICP.

### EXAMPLE 4

516 ppm of dibutyltin dilaurate (corresponds to 100 ppm of Sn) were added before the beginning of the reduced-pressure phase to a polyesterol obtained from 59.36 kg of ADA and 40.6 kg of B14. The reactivity of the sample was determined immediately after the end of the reaction. Dried compressed air (50 l/h) was passed into about 4 kg of the sample in a 5 l three-necked flask having a stirrer and gas inlet tube at 95° C. A further 4 kg of the sample was stored in a flask under reduced pressure (10 mbar) at 95° C. or in a drying oven at 95° C. The reactivity of the samples was determined at regular intervals. The table below shows that the reactivity of the samples stored with introduction of air decreases whereas the reactivity of the samples stored under reduced pressure remains virtually constant.

TABLE

| | | | |
|---|---|---|---|
| Storage time | RN | Sn (polarography) | Sn (ICP-AAS) |

Storage under introduction of air at 95° C.

| | | | |
|---|---|---|---|
| 0 h | 2.3 K/s | 33.2 ppm | 111 ppm |
| 24 h | 1.9 K/s | 32.5 ppm | 113 ppm |
| 48 h | 1.8 K/s | 33.4 ppm | 112 ppm |
| 116 h | 0.7 K/s | 12.2 ppm | 112 ppm |
| 140 h | 0.4 K/s | 5.2 ppm | 111 ppm |

Storage under reduced pressure at 95° C.

| | | | |
|---|---|---|---|
| 0 h | 2.3 K/s | 33.2 ppm | 111 ppm |
| 24 h | 2.1 K/s | 33.1 ppm | 110 ppm |
| 48 h | 1.9 K/s | 31.0 ppm | 113 ppm |
| 116 h | 1.9 K/s | 32.8 ppm | 111 ppm |
| 140 h | 1.8 K/s | 27.0 ppm | 114 ppm |

Storage in a drying oven at 95° C.

| | | | |
|---|---|---|---|
| 0 h | 2.3 K/s | 33.2 ppm | 111 ppm |
| 24 h | 1.8 K/s | 36.1 ppm | 110 ppm |
| 48 h | 1.6 K/s | 34.0 ppm | 112 ppm |
| 116 h | 1.5 K/s | 34.5 ppm | 110 ppm |
| 140 h | 1.4 K/s | 26.8 ppm | 111 ppm |

The example shows that the reactivity of Sn(IV)-catalyzed polyols decreases as a result of passing in air. With the decrease in the reactivity, the polarographically detectable tin content decreases.

### EXAMPLE 5

454 ppm of tin 2-ethylhexanoate were added before the beginning of the reduced-pressure phase to a polyesterol obtained from 59.36 kg of ADA and 40.6 kg of B14. The reactivity of the sample was determined immediately after the end of the reaction. Dried compressed air was passed into about 4 kg of the sample in a 5 l three-necked flask having a stirrer and gas inlet tube at 95° C. A further 4 kg of the sample was stored in a flask under reduced pressure (10 mbar) at 95° C. or in a drying oven at 95° C. The reactivity of the samples was determined at regular intervals. The table below shows that the reactivity of the samples stored with introduction of air decreases whereas the reactivity of the samples stored under reduced pressure remains constant.

TABLE

| | | | |
|---|---|---|---|
| Storage time | RN | Sn (polarography) | Sn (RFA) |

Storage under introduction of air at 95° C.

| | | | |
|---|---|---|---|
| 0 h | 1.2 K/s | 41 ppm | 133 ppm |
| 24 h | 0.3 K/s | 5 ppm | 132 ppm |
| 48 h | 0.3 K/s | <1 ppm | 119 ppm |
| 118 h | 0.3 K/s | <1 ppm | 132 ppm |

Storage under reduced pressure at 95° C.

| | | | |
|---|---|---|---|
| 0 h | 1.2 K/s | 41 ppm | 133 ppm |
| 24 h | 1.4 K/s | 45 ppm | 127 ppm |
| 48 h | 1.5 K/s | 45 ppm | 119 ppm |
| 118 h | 1.5 K/s | 50 ppm | 119 ppm |

Storage in a drying oven at 95° C.

| | | | |
|---|---|---|---|
| 0 h | 1.2 K/s | 41 ppm | 133 ppm |
| 24 h | 1.3 K/s | 43 ppm | 132 ppm |
| 48 h | 1.4 K/s | 43 ppm | 117 ppm |
| 118 h | 0.5 K/s | 34 ppm | 134 ppm |

1. A process for the preparation of polyesterols of reduced reactivity by polycondensation of dicarboxylic acids/dicarboxylic acid derivatives and H-functional substances using tin catalysts, wherein the polyesterols are brought into contact with oxygen after the end of the esterification reaction.

2. The process as claimed in claim 1, wherein atmospheric oxygen is used.

3. The process as claimed in claim 1, wherein the contact with air is effected by passing in dry compressed air.

4. The process as claimed in claim 1, wherein the contact with air is effected by passing in a lean air mixture.

5. The process as claimed in claim 1, wherein air is passed into the polyester reactor after the end of the reduced-pressure phase.

6. The process as claimed in claim 1, wherein air is passed into the polyesterol storage tank.

7. The process as claimed in claim 1, wherein air is passed into the tanker vehicles which are used for transporting the polyesterols.

8. A polyesterol prepared by the process as claimed in claim 1.

9. The use of a polyesterol as claimed in claim 8 for the preparation of polyurethanes.
